(19) United States
(12) Patent Application Publication
(10) Pub. No.: US 2012/0195856 A1
(43) Pub. Date: Aug. 2, 2012

(54) Method of treating patients with cardiovascular illness

(76) Inventors: Artur Martynov, Kharkov (UA); Boris S. Farber, Brooklyn, NY (US); Sonya Sophya Farber, New York, NY (US)

(21) Appl. No.: 12/931,464

(22) Filed: Feb. 1, 2011

(51) Int. Cl. A61K 38/21; A61K 39/395; A61P 9/00; A61K 31/522

(52) U.S. Cl. 424/85.7; 514/263.37; 424/159.1

(57) ABSTRACT

This invention may be used in human and veterinary medicine in combination with traditional methods of treatment of cardiovascular illnesses for the purpose of increasing their effectiveness.

A method of treating patients with cardiovascular illnesses, distinct in that an anti-viral drug is used as an additional component of standard treatment before the beginning of and in parallel with standard treatment, in which Acyclovir, Valacyclovir, alpha interferon, a specific antiviral immunoglobulin or a combination of these substances are used as an anti-viral drug, which may be used for inclusion in the standard scheme of treatment of patients with cardiovascular diseases.

The proposed method allows us to significantly improve the results of the treatment of patients with cardiovascular system pathology, decrease the number of relapses, improve patients' quality of life, and significantly extend their lives. Because the proposed drugs are already approved for use, there are no technical difficulties facing their inclusion in the treatment plans of patients with cardiovascular system pathology.

### TECHNICAL FIELD

This invention is related to medicine—specifically, to cardiology—and is intended to increase the effectiveness of the treatment of cardiovascular pathologies in humans.

### Previous Level of Technology

Cardiovascular diseases (including atherosclerosis and its complications) are a major cause of death for people in developed countries [1]. For Ukraine, this problem is even more pressing: according to the WHO, in Ukraine in 2005, cardiovascular disease (CVD) was the reason for approximately 60% of all deaths, which significantly exceeds the analogous indicator in the developed countries of Europe (38%) [2]. Therefore, any new information on the mechanisms of development for atherosclerosis, as well as the development of new, more effective methods for its treatment, are of momentous social importance.

In accordance with the modern concept of medical science, atherosclerosis is a chronic inflammatory disease of the walls of the large arteries [3, 4, 5]. In atherosclerosis, lipids, fibroid elements, and calcium salts collect in the arteries, which leads to a decrease in the clearance of the arteries and in their elasticity. In principle, this process begins almost at birth and accompanies the inevitable process of the body's aging [6, 7, 8]. The development of atherosclerosis begins with damage to the endothelium of blood vessels and the disruption of its functions. The damaged wall of the blood vessel, in addition to a local compensatory reaction, initiates a powerful systemic response in the form of a cascade of molecular reactions and cell processes such as an expression of adhesion molecules, hemotaxis factors, inflammatory cytokines, and growth factors, involvement of leukocytes, expression by the bone marrow of progenitor cells and their pickup by the vessel wall to eliminate the damage, and so on [9]. Normally, this process leads to the restoration of the function of the endothelium and homeostasis; however, in many cases, when an unfortunate combination of risk factors comes together, the process may become pathological, leading to a destructive systemic inflammatory reaction.

A few risk factors are known, both genetically conditioned and external in nature, that cause damage to the endothelium and activate atherogenesis: an increased cholesterol level (in part, the low-density lipoproteins, LDLs), an increased level of homocysteine, arterial hypertension, diabetes, smoking, genetic disposition, and age (aging). The process of atherosclerosis is a long and complex one; several detailed overview articles are dedicated to the topic [10]. The following stages may be determined in the development of atherosclerosis.

Stage I: Fatty streaks: aggregation of LDL particles in the subendothelial layer (intima) of the blood vessel. LDL is subject to acidification (the process is stimulated by free radicals), and in that form, the lipoprotein penetrates to the intima more actively. LDL plays a key role in this process: the higher its level, the faster atherogenesis takes place. Further, with the active participation of inflammation mediators (interleukine-1, cachectin a, growth factures, adhesion molecules), an active migration of monocytes and T-lymphocytes to the location of the microscopic damage to the endothelium takes place. They penetrate to the intima, the monocytes turn into macrophages, and the macrophages pick up the acidized LDL. Thus foam cells are formed. The sections of the massive aggregation of foam cells in this stage can be visualized as fatty streaks.

Stage II: Formation of a necrotic core. With time, the foam cells die off, and their lipoprotein-containing remains collect in the intima and as a result form a necrotic core of atherosclerotic disease. Sometimes smooth muscle cells (SMCs) embed themselves in the fatty streaks, migrating from the medial layer of the blood vessel.

Stage III: Intracellular aggregation of lipoprotein. The acidified LDL continues to aggregate in the intima; the atherosclerotic plaque grows in size.

Stage IV: Formation of a lipid core. The buildup of SMCs continues; in the process of the aggregation of lipoprotein in the intima, calcification becomes involved, taking place first inside the SMCs, and after their death, between the muscle wall of the blood vessel and the external part of the atherosclerotic plaque.

Stage V: Formation of an atheroma. Between the fat deposit and the intima, a protective layer forms, consisting primarily of fibrin and collagen filaments. This encapsulation of the fat deposit is called an atheroma. For a certain amount of time, the atheroma grows inside the vessel wall, causing a compensatory expansion of the vessel, but after it reaches a critical size, it begins to encroach on the vessel's opening, decreasing its diameter and affecting blood flow; stenosis is developed. It should be noted that before this stage (and later, in many cases) the process of the development of atherosclerosis goes on asymptomatically and lacks almost all clinical manifestation; only stenosis of more than 77% is considered to be the limit in cardiology after which a clinically expressed disease begins.

Stage VI: Rupture of the atheroma and thrombosis. When the integrity of the fibral capsule covering the atheroma is breached, thrombocytes and tissue factors are set free, leading to a cascade of biochemical blood clotting reactions. A clot is thus formed. Whereas the duration of the previous stages of atherosclerotic development may have been from several weeks to decades, the creation of the clot may occur within scant minutes, leading to an embolism and often causing catastrophic consequences in the form of a heart attack or stroke. According to clinical study data, only about 14% of clinically expressed phenomena occur at a compression of the blood vessel openings of 77% and higher; the majority of life-threatening and deadly events occur as the result of the abruption of the plaque and the following thrombosis [11].

Infectious components in the etiology of atherosclerosis. Notwithstanding the fact that the risk factors of atherosclerosis are well-known these days, even all of them together cannot explain half of the clinical cases of atherosclerosis [12]. Thus infectious diseases are being entered in the list of risk factors with increasing certainty. The infectious etiology was confirmed for many chronic illnesses, such as stomach ulcers (Heliobacter pylori), cervical cancer (various papilloma viruses) and liver cancer (the hepatitis B and C viruses). Increasing amounts of data indicate the participation of infection in the etiology of diabetes mellitus, Alzheimer's disease, various neurological diseases, and cardiovascular disease [13]. The hypothesis on the infectious nature of atherosclerosis first appeared in the middle of the 19th century in the works of R. Virchow [14], and later, in 1889, in the works of Gilbert and Lion. The association of atherosclerosis and viral infection was empirically grounded in the 1970s in the works of C. G. Fabricant et al, in which atherosclerosis-like changes in the blood vessels developed in chicks infected with the Marek's disease virus, an avian variant of the herpes virus [15]. Many viruses have been proposed in the role of possible pathogens in atherogenesis: cytomegalovirus (CMV) [16, 17, 18, 19, 20, 21, 22], herpes simplex (HSV) [23, 24] and Epstein-Barr [25, 26] and many others. The majority of experimental studies of the association of infections and atherosclerosis have been dedicated to Ch. pneumoniae and cytomegalovirus. Both of these microorganisms are highly likely to be found in vessels with atherosclerosis and atherosclerotic plaques, and being seropositive for them correlates well with atherosclerosis and severe courses of cardiovascular disease [27, 12, 28].

Regardless of the fact that the association of two infectious agents—Ch. pneumoniae and cytomegalovirus—with cardiovascular diseases can be considered proven, this does not indicate a cause and effect connection. Moreover, Koch's classic postulates cannot be fulfilled in the case of a multi-factor disease like atherosclerosis [1, 31]. Most likely, the infections are neither necessary nor a sufficient reason for the development of atherosclerosis, but they are a risk factor that increases the likelihood of the development of the pathology. In order to prove that infections participate in atherosclerosis, in addition to the histopathological data, a precise concept of the mechanism(s) of the pathogenesis, experimentally confirmed in vitro and in vivo, and as a result, of the methods of treatment, the effectiveness of which has been proven in experiment and in clinical conditions, are needed. Many mechanisms of pathogenic activity of infectious agents on the cardiovascular system are known [29]:
an increase in the proliferation of HM cells and in their migration (human cytomegalovirus)
protection of the cells of the endothelium from apoptosis, which leads to their excess aggregation and an increase in the size of the atherosclerotic plaque (cytomegalovirus, Ch. pneumoniae)
increased speed of lipid aggregation (cytomegalovirus, Ch. pneumoniae)
an increase in the pro-coagulation activity of the endothelial cells (cytomegalovirus, HSV)
an increase in the expression of cytokines, chemokines, and adhesion molecules, an appearance of severe phase proteins (C-reactive protein, serum amyloid C, etc.), which leads to a vicious cycle of hyperergic inflammatory reaction, and, as a result, to damage to the endothelium (nearly all pathogenic microorganisms are suspected)
an increase in the level of reactive forms of oxygen (oxygen ions, free radicals, peroxides) caused by increased acidification of LDL (cytomegalovirus)
autoimmune reactions; certain proteins created by pathogens that are homologous to proteins found in the human body, which may be the reason for the arousal of an autoimmune reaction, a high level of homologic proteins are seen for the Heat Shock Protein (HSP) of various bacteria and humans (HSP60)

A good deal of data obtained in animal experiments confirms the results of the histopathology and in vitro research. According to the data of C. G. Fabricant et al [15], the Marek's disease virus caused atherosclerosis in chicks, and the immunization of healthy animals prevented the development of the pathology regardless of the amount of cholesterol in the feed taken. They also studied the effect of immune stimulation therapy on the development of atherosclerosis in rabbits experimentally infected with HSV: a significant slowing in the atherosclerosis development process was noted [16]. A significant amount of data obtained as a result of histopathological analysis as well as in vitro and in vivo in the experiment confirms the hypothesis of the infectious component in the etiology of atherosclerosis.

The next stage in the development of the concept of infectious components in the etiology of atherosclerosis was clinical trials with the use of antibiotics. Since the majority of the experimental data bore witness to the involvement of Ch. pneumoniae in atherosclerosis, the majority of the experiments were directed at eliminating it. The most effective medicines against Ch. pneumoniae are believed to be antibiotics from the macrolide group, including azithromycin, due to their ability to penetrate inside the cell and act on intracellular parasites [30, 31]. From 1997 through 2005, many clinical trials were conducted using azithromycin (or other macrolides: roxithromycin and clarithromycin) as one of the agents for the compound treatment of cardiovascular disease (ischemic heart disease, old myocardial infarction, and unstable stenocardia) [32, 33, 34, 35]. Out of the more than 100 studies, eleven were promising, randomized, and placebo-controlled. In them, the effect of antibiotic therapy on the frequency of appearance of myocardial infarction and stroke, their repeated development, and the overall mortality rate as a result of cardiovascular disease were evaluated [33]. A comparison of the results of the studies held is a difficult task due to the variations in the criteria for the inclusion of patients, the design of the studies themselves, and the duration of observation after treatment. Even so, a conclusion may be drawn from the results of an analysis of the studies: notwithstanding the fact that after the first pilot studies, encouraging results were achieved, more large-scale clinical studies did not demonstrate any advantage whatsoever to antibiotic therapy in the treatment of cardiovascular disease.

Atherosclerosis is a widespread chronic disease with a multi-factor and not fully explained etiology and a long and secret pathogenesis. The evolution of the understanding of the reasons for and mechanisms of this disease's appearance by modern science is developing quickly, although it is insufficient to the demands of the times. In the 1990s, it was hoped that cardiovascular diseases could be eliminated by the end of the century through control over cholesterol and arterial pressure; ten years later, it became clear that this optimistic prognosis needed to be reexamined [10]. Thus a change in paradigms occurred, as the result of which atherosclerosis ceased to be a simple disruption to the metabolism by cholesterol and was recognized as an inflammatory disease [36, 10]. Traditional and prospective methods for the treatment of atherosclerosis through drugs are directed toward various links in the pathogenetic chain, such as lipid exchange (statins, fibrates, cholesterol absorption inhibitors), blockade of renin-angiotensin systems (angiotensin converting enzyme inhibitors [ACEI]), blockade of 3-adrenoreceptors and excess calcium accumulation (calcium ion antagonists), oxidative stress (tocopherol analogues: vitamin E, probucol, AGI-1067), excess proliferation of HIM cells (HM cell growth inhibitor), aggregation of thrombocytes (thrombocyte activation inhibitors), inflammatory processes in the blood vessels (acetosalicylic acid, AGI-1067), and thrombosis (anti-thrombotic drugs, heparin, thrombin antagonists). The most effective are the drugs with a pleiotropic effect: that is, those that act simultaneously on various mechanisms of pathogenesis such as statins (lipid exchange, antiatherosclerotic, anti-inflammatory, and anti-thrombolytic action) and the new class of therapeutic agents, vascular protectants, which have antioxidant and anti-inflammatory properties and also decrease post-angioplasty restenosis [37].

In addition, even in a complex approach and with the use of new pleiotropic drugs, the treatment of atherosclerosis remains, as before, pathogenetic. It is possible that the next big moment in the evolution of atherosclerosis and cardiovascular disease treatment will be the reexamination of the concept of the therapy and a transition from pathogenetic to etiotropic and pathogenetic treatment, as well as to preventative treatment (in the early stages before the manifestation of clinical symptoms), with the goal of preventing the development of severe forms of atherosclerosis. One of the types of this therapy could be treatment of infections that are risk factors for the development of atherosclerosis (Ch. pneumoniae, HSV, and CMV). Interferon and its inducers have been effective in the treatment of viral myocardias of various etiologies caused by entero- and adenoviruses, as well as by herpes viruses [38]. These in vitro studies are promising; however, in order to implement atherosclerosis therapy with the use of interferons, large-scale clinical trials are needed, as well as determination of groups of patients and stages of illness for which this therapy would be most effective.

The results of many studies indicate that infectious diseases heavily influence the etiology and pathogenesis of atherosclerosis, but data on the supposed causes of the diseases are contradictory. Considering that atherosclerosis is a multi-factor disease, Koch's postulates are not applicable in this case; most likely a single infection will not be discovered that is a trigger for atherosclerosis. The most likely risk factor is the “infection load” factor. Data from many studies indicate a direct correlation between the number of infections discovered in a single patient and the level of severity of cardiovascular diseases, as well as mortality due to cardiovascular diseases.

Clinical studies conducted using antibiotics did not facilitate a decrease in the likelihood of infarction and mortality due to cardiovascular diseases. There may be many reasons for this, but most likely, it is that the activity of the antibiotics was directed toward one infection only, while the main role in the development of atherosclerosis is played by herpesvirus infections.

Use of the most accessible tablet form of the herpes drug Valacyclovir and its injectible counterpart in complex therapy for the diseases caused by atherosclerosis may significantly improve the prognosis of the course of the disease, increase remission periods, and prevent relapses of heart attacks and strokes.

A method of diagnosis, prevention, and therapy of restenosis/atherosclerosis is known that comprises a combination of cytomegalovirus diagnosis in the patient's body and the following therapeutic vaccination of patients against cytomegalovirus infection [39]. Genotyping of the histocompatibility complex is also used in the diagnostic method. The shortcoming of this method is that it does not take into account other herpes viruses in the development of atherosclerosis: herpes types 1 and 2, the Epstein-Barr virus, and herpes type 6. Also, the developers did not propose an effective scheme for the treatment of atherosclerosis; vaccination of the body in the presence of a large quantity of cytomegalovirus antigens has little effect, and specific immunity is not persistent. Thus the effect from the proposed scheme of atherosclerosis prevention will not be persistent. Moreover, the developers proposed a complex vaccination scheme, not only against the cytomegalovirus antigen, but also to the p53 cell protein, which may lead to the development of autoimmune reaction to the body's own tissues.

### Disclosure of the Invention

The task of the invention is to increase the effectiveness of the method of treating cardiovascular diseases with account taken of the participation of the herpes virus in the etiology and pathogenesis of these diseases.

The task set is addressed through inclusion of anti-viral drugs in the standard treatment scheme of cardiovascular diseases. Acyclovir drugs are used (injected and tablet Acyclovir and Valacyclovir) other anti-viral substances (specific antiviral immunoglobulins and alpha-interferon) before, after, and in combination with the standard (surgical and chemotherapeutic) treatment of cardiovascular diseases. Valacyclovir is taken in 1-2 g doses 3-4 times a day from 7-20 days in 3-7 courses. Acyclovir is administered intravenously in 0.5-1.0 g doses 3-4 times a day from 7-20 days in 3-7 courses as well. This combination will lead to the rehabilitation of the immune system, expand the spectrum of standard treatment methods, significantly improve the effectiveness of patient treatment, prevent relapses in patients with unstable stenocardia, normalize the level of lipids and cholesterol in the blood, and facilitate long-term stable remission in cardiovascular diseases.

### EXAMPLES OF INVENTION IMPLEMENTATION

Example 1. Patient L., 52 years old, came to the clinic with a diagnosis of ischemic heart disease. Exertional angina, functional class III. Postinfarction cardiosclerosis (myocardial infarction in 1981). Atherosclerosis of the coronary arteries. Circulatory deficiency stage IIA. Cerebral atherosclerosis. Chronic cerebral impairment. Symptomatic hypertension. At arrival, complained of a squeezing pain in the area of the heart with radiation to the right hand, which arise both at rest and during physical activity (up to 4-5 times per day) and were relieved by taking nitroglycerin (up to 8 times per day), as well as shortness of breath while walking, vertigo, periodic headaches, ringing in the ears, irritability, and insomnia. The patient was prescribed treatment: anti-angina drugs (Nitrosorbidum 30 mg/day, Corinfar 30 mg/day; also has hypotensive activity), and Valacyclovir 2 tablets (1.0 g) three times a day. As a result of the treatment, a decrease in the blood serum of total cholesterol from 6.71 mmol/l to 3.26 mmol/l, of triglycerides from 3.4 mmol/l to 1.04 mmol/l, of b-lipoproteins from 770 conditional units to 310 conditional units, cholesterol/LDL from 3.99 mmol/l to 3.68 mmol/l, and LDL from 1.55 mmol/l to 0.47 mmol/l. The atherogenesis index fell from 4.74 to 3.38, and there was an increase in cholesterol/HDL from 1.17 mmol/l to 1.20 mmol/l. During the treatment process, a slowing in the free-radical acidization of lipids was seen; the content of serum malondialdehyde fell from 0.50 mcmol/ml to 0.31 mcmol/ml, diethynoid conjugates fell from 1.332 mcmol/ml to 0.70 mcmol/ml, triethynoid conjugates declined from 0.23 mcmol/ml to 0.14 mcmol/ml; the a-tocopherol content recovered from 2.69 mcmol/ml to 4.32 mcmol/ml; the glutathione reductase activity went from 12.4 mcmol/l h to 22.0 mcmol/l h. On the electrocardiogram, a decrease in the T index from 35.0 to 0, in the SST from 11.0 to 0, and in the NST from 10.0 to 0 were seen. Hemodynamic indicators also improved; the total peripheral vascular resistance decreased from 2917.9 dyn s cm⁻⁵ to 2673.6 dyn s cm⁻⁵; the specific peripheral resistance went from 74.53 conditional units to 65.97 conditional units; the energy loss fell from 15.96 conditional units to 10.37 conditional units; the flow rate increased from 126.5 ml/sec to 169.71 ml/sec, and the capacity of the left ventricle improved from 2.02 W to 2.29 W. Within seven weeks, the patient's condition had improved significantly. Angina pain and shortness of breath not present; nitroglycerine tablets not used. Vertigo and headaches had decreased.

Example 2. Patient M., 60 years old. Diagnosis: ischemic heart disease: Postinfarction cardiosclerosis (1999). Stage 3 hypertonic disease. Stage-1 circulatory deficiency. Treatment: Corvitol 50 mg 2×/day, Renitec 10 mg 2×/day, aspirin 80 mg/day, Zocor 10 mg in the evening. After six months of simvastatin 10 mg/day, the following indicators of the lipid and liver complexes were obtained: total cholesterol 4.9 mmoles/l (goal indicator 5.0 mmoles/l), triglycerides 1.55 mmoles/l, GOT 110 IU/l (norm 10-39 IU/l), GPT 100 IU/l (norm 10-35 IU/l). After Valacyclovir 2 tablets (1.0 g) 3×/day for 3 courses of 7 days, the following indicators of the lipid and liver complexes were obtained: total cholesterol 4.7 mmoles/l (goal value 5.0 mmoles/l), triglycerides 1.3 mmoles/l, GOT 22 IU/l (norm 10-39 IU/l), GPT 32 IU/l (norm 10-35 IU/l).

Example 3. Patient Ch., 40 years old. Diagnosis: ischemic heart disease: Postinfarction cardiosclerosis (1998). Stage 1 circulatory deficiency. Treatment: metoprolol (Corvitol) 25 mg 2×/day, aspirin 80 mg/day, simvastatin (Zocor) 10 mg in the evening. After five months of simvastatin 10 mg/day, the following indicators of the lipid and liver complexes were obtained: total cholesterol 5.6 mmoles/1 (goal indicator 5.0 mmoles/l), triglycerides 2.55 mmoles/l, GOT 89 IU/l (norm 10-39 IU/l), GPT 96 IU/l (norm 10-35 IU/l). After Valacyclovir 1 g 3×/day for 5 courses of 7 days each with 7-day intervals, the following indicators of the lipid and liver complexes were obtained: total cholesterol 4.9 mmoles/l, triglycerides 1.95 mmoles/l, GOT 38 IU/l (norm 10-39 IU/l), GPT 30 IU/l (norm 10-35 IU/l). As the examples provided indicate, the combination of a standard treatment scheme with Valacyclovir with an increase in the level of transaminase while statins are being taken allows an increase in the effectiveness of atherosclerosis and dyslipidemia treatment.

Thus in the process of treatment, in the patient who took Valacyclovir, lipidogram, peroxide acidization of lipids, electrocardiography, and tetrapolar rheography indicators normalized. Valacyclovir side effects were not observed.

Administering according to a plan of fewer than 3 courses of seven days each of less than 1 g 3 times a day for Valocyclovir perorally and Acyclovir at a dosage of less than 0.5 g 2 times a day in injected form does not provide long-term remission of the illness or a substantial positive stabilization of the patient's condition. The use of the antiviral drug is not useful for a longer period, as the remission period begins for the patient and the drug is ineffective.

Example 4. Patient L., 48 years old. Diagnosis: ischemic heart disease: Postinfarction cardiosclerosis (2006). Stage 3 hypertonic disease. Stage 1 circulatory deficiency. Treatment: Corvitol 50 mg 2×/day, Renitec 10 mg 2×/day, aspirin 80 mg/day, Zocor 20 mg in the evening. After six months of Zocor 20 mg/day, the following indicators of the lipid and liver complexes were obtained: total cholesterol 3.9 mmoles/l (goal indicator 4.0 mmoles/l), triglycerides 1.4 mmoles/l, GOT 67 IU/l (norm 10-39 IU/l), GPT 88 IU/l (norm 10-35 IU/l). As a result of IIF study of the blood immunocytes, antigens to the Epstein-Barr virus, cytomegalovirus, and herpes type 1 virus were found.

The patient then underwent combination therapy with anti-viral drugs: immunoglobulin to treat the EBV at a dosage of 13 ml of a 10% solution immediately (7 ml per injection) intramuscularly once a week three times; Valacyclovir to treat the CMV at a dosage of 2 tablets (1.0 g) 3 times a day in three courses of seven days each; Laferobion 3 million IU once per day for seven days in a row. The following indicators were obtained: total cholesterol 1.5 mmoles/l, triglycerides 1.3 mmoles/l, GOT 20 IU/l (norm 10-38 IU/l), GPT 35 IU/l (norm 10-35 IU/l). Repeat IIF study after two months did not discover antigens to any of the viruses discovered earlier in the immunocytes.

### INDUSTRIAL APPLICABILITY

This invention is related to medicine—specifically to cardiology—and may be used in hospitals' cardiology departments and for the treatment of ambulatory patients for inclusion in the treatment complex with the goal of increasing the effectiveness of treatment. All the proposed components are produced by the pharmaceutical industry and are accessible for use.

### REFERENCES

1 Braunwald E. Cardiovascular Medicine at the Turn of the Millennium: Triumphs, Concerns, and Opportunities//N. Engl. J. Med.—1997.—Vol. 337, N 19.—P. 1360-1369.
2 Highlights on Health in Ukraine//WHO Regional Office for Europe.—2005.—http:// www.euro.who.int/document/e88285.pdf
3 Goldschmidt-Clermont P. J., Creager M. A., Lorsordo D. W. et al. Atherosclerosis 2005: Recent Discoveries and Novel Hypotheses//Circulation.—2005.—Vol. 112, N 21.—P. 3348-3353.
4 Lusis A. J. Atherosclerosis//Nature.—2000.—Vol. 407, N 6801.—P. 233-241.
5 Ross R. Atherosclerosis—An Inflammatory Disease//N. Engl. J. Med.—1999.—Vol. 340, N 2.—P. 115-126.
6 Lusis A. J. Atherosclerosis//Nature.—2000.—Vol. 407, N 6801.—P. 233-241.
7 Rauscher F. M., Goldschmidt-Clermont P. J., Davis B. H. et al. Aging, Progenitor Cell Exhaustion, and Atherosclerosis//Circulation.—2003.—Vol. 108, N 4.—P. 457-463.
8 Serrano A. L., Andres V. Telomeres and Cardiovascular Disease: Does Size Matter?//Circ. Res—2004.—Vol. 94, N 5.—P. 575-584.
9 Goldschmidt-Clermont P. J., Creager M. A., Lorsordo D. W. et al. Atherosclerosis 2005: Recent Discoveries and Novel Hypotheses//Circulation.—2005.—Vol. 112, N 21.—P. 3348-3353.
10 Hansson G. K. Inflammation, Atherosclerosis, and Coronary Artery Disease//N. Engl. J. Med.—2005.—Vol. 352, N 16.—P. 1685-1695.
11 http://en.wikipedia.org/wiki/Atherosclerosis
12 Epstein S. E., Zhou Y. F., Zhu J. Infection and Atherosclerosis: Emerging Mechanistic Paradigms//Circulation.—1999.—Vol. 100, N 4.—P. 20-28.
13 Stacey L. K., Lemon S. M., Najafi M., Editors The Infectious Etiology of Chronic Diseases: Defining the Relationship, Enhancing the Research, and Mitigating the Effects—Wash., D.C.: The National Academic Press, 2004.—215 p.
14 Virchow R. Der atheromatose prozess der arterien//Wien. Med. Wochenschr.—1856.—Vol. 6.—P. 809-812, 825-827
15 Fabricant C. G., Fabricant J., Litrenta M. M., Minick C. R. Virus-induced atherosclerosis//J. Exp. Med.—1978.—Vol. 148, N 1.—P. 335-340.
16 Gandzha I M. Viral infection, atherosclerosis, and ischemic heart disease//Lik. sprava. Vracheb. delo.—2001.—Ns 1.—pp. 65-67.
17 Bruggeman C. A. Does Cytomegalovirus Play a Role in Atherosclerosis?//Herpes.—2000.—Vol. 7, N 2.—P. 51-54.
18 Grahame-Clarke C. Human cytomegalovirus, endothelial function and atherosclerosis //Herpes.—2005.—Vol. 12, N 2.—P. 42-45.
19 Grahame-Clarke C, Chan N. N, Andrew D. et al. Human cytomegalovirus seropositivity is associated with impaired vascular function//Circulation.—2003.—Vol. 108, N 6.—P. 678-683.
20 Reinhardt B., Minisini R., Mertens T Opinion article: cytomegalovirus is a risk factor in atherogenesis//Herpes.—2002.—Vol. 9, N 1.—P. 21-23.
21 Schlitt A., Blankenberg S., Weise K. et al. Herpesvirus DNA (Epstein-Barr virus, herpes simplex virus, cytomegalovirus) in circulating monocytes of patients with coronary artery disease//Acta Cardiol.—2005.—Vol. 60, N 6.—P. 605-610.
22 Sorlie P. D., Adam E., Melnick S. L. et al. Cytomegalovirus/herpesvirus and carotid atherosclerosis: the ARIC Study//J. Med. Virol.—1994.—Vol. 42, N 1.—P. 33-37.
23 Kotronias D. F., Kapranos N. Herpes simplex virus as a determinant risk factor for coronary artery atherosclerosis and myocardial infarction//In Vivo.—2005.—Vol, 19, N 2.—P. 351-357.
24 Visser M. R., Vercellotti G. M. Herpes simplex vims and atherosclerosis//Eur. Heart J.—1993.—Vol. 14 Suppl K.—P. 39-42.
25 Morre S. A., Stooker W., Lagrand W. K et al. Microorganisms in the aetiology of atherosclerosis//J. Clin. Pathol.—2000.—Vol. 53, N 9.—P. 647-654.
26 Schlitt A., Blankenberg S., Weise K. et al. Herpesvirus DNA (Epstein-Barr virus, herpes simplex virus, cytomegalovirus) in circulating monocytes of patients with coronary artery disease//Acta Cardiol.—2005.—Vol. 60, N 6.—Pp. 605-610.
27 Dunne M. Infectious Agents and Cardiovascular Disease//The Infectious Etiology of Chronic Diseases: Defining the Relationship, Enhancing the Research, and Mitigating the Effects—Wash., D.C: The National Academic Press, 2004.—215 p.
28 Reinhardt B., Minisini R., Mertens T Opinion article: cytomegalovirus is a risk factor in atherogenesis//Herpes.—2002.—Vol. 9, N 1.—P. 21-23.
29 Vatutin N T, Chupina V A. Infection as a factor in the development of atherosclerosis and its complications//Cardiology.—2000.—No. 2.—pp. 67-71.
30 Lindholt J. S., Stowing J., Andersen P. L. et al. A review of macrolide treatment of atherosclerosis and abdominal aortic aneurysms//Curr. Drag. Targets Infect. Disord.—2003.—Vol. 3, N 1.—P. 55-63.
31 Pechere J. C. New perspectives on macrolide antibiotics//Int. J. Antimicrob. Agents.—2001.—Vol. 18, Suppl 1.—P. S93-97.
32 Anderson J. L., Muhlestein J. B. Antibiotic trials for coronary heart disease//Tex. Heart. Inst. J.—2004.—Vol. 31, N 1.—P. 33-38.
33 Andraws R., Berger J. S., Brown D. L. Effects of antibiotic therapy on outcomes of patients with coronary artery disease: a meta-analysis of randomized controlled trials//JAMA.—2005.—Vol. 293, N 21.—P. 2641-2647.
34 Kuppuswamy V. C, Gupta S. Antibiotic therapy for coronary heart disease: the myth and the reality//Timely Top. Med. Cardiovasc. Dis.—2006.—Vol. 10.—P. E2.
35 Muhlestein J. B. Antibiotic treatment of atherosclerosis//Curr. Opin. Lipidol.—2003.—Vol. 14, N 6.—P. 605-614.
36 Goldschmidt-Clermont P. J., Creager M. A., Lorsordo D. W. et al. Atherosclerosis 2005: Recent Discoveries and Novel Hypotheses//Circulation.—2005.—Vol. 112, N 21.—P. 3348-3353
37 Meng C. Q., Somers P. K, Rachita C. L. et al. Novel phenolic antioxidants as multifunctional inhibitors of inducible VCAM-1 expression for use in atherosclerosis//Bioorg. Med. Chem. Lett.—2002.—Vol. 12, N 18.—P. 2545-2548.
38 Cull V. S., Bartlett E. J., James C M. Type I interferon gene therapy protects against cytomegalovirus-induced myocarditis//Immunology.—2002.—Vol. 106, N 3.—P. 428-437.
39 U.S. Pat. No. 6,183,752 Restenosis/atherosclerosis diagnosis, prophylaxis and therapy

1. A method of medical treatment for patients with atherosclerosis complications, the method comprising a standard atherosclerosis treatment in combination with administration of at least one of anti-herpetic drugs.

2. A method of medical treatment for patients with atherosclerosis complications of claim 1, wherein the anti-herpetic drug is Valacyclovir.

3. A method of claim 2, wherein Valacyclovir is administered in 3 to 7 courses of 7 to 20 days each, in doses of 1-2 g, 3-4 times per day, with intervals of 7-20-days between consecutive courses.

4. A method of claim 1 wherein the intravenously injectible form of Acyclovir is used as an anti-herpetic drug.

5. A method of claim 4, wherein Acyclovir is administered in 3-7 courses of 7-20 days each in dosages of 0.5-1 g, 1-4 times a day, with intervals of 7-20 days between consecutive courses.

6. (canceled)

7. A method of claim 1, wherein the anti-herpetic drug is a specific anti-herpetic immunoglobulin in injected form.

8. A method of claim 1, wherein the anti-herpetic drug, is a combination of drugs. Valacyclovir, Acyclovir, antiCMV, antiEBV, antiHSV1, antiHSV2 and antiHZV

9. A method of claim 7, wherein the specific anti-herpetic immunoglobulin in injected form is at least one of antiCMV, antiEBV, antiHSV1, antiHSV2 or antiHZV
